(19) United States
(12) Patent Application Publication
(10) Pub. No.: US 2023/0363695 A1
(43) Pub. Date: Nov. 16, 2023

(54) CLASSIFICATION OF FUNCTIONAL LUMEN IMAGING PROBE DATA

(71) Applicant: Northwestern University, Evanston, IL (US)

(72) Inventors: Mozziyar Etemadi, Evanston, IL (US); John E. Pandolfino, Evanston, IL (US); Dustin Allan Carlson, Evanston, IL (US); Wenjun Kou, Evanston, IL (US); Matthew William Klug, Evanston, IL (US); Priyanka Soni, Evanston, IL (US)

(21) Appl. No.: 18/245,324

(22) PCT Filed: Sep. 15, 2021
(86) PCT No.: PCT/US2021/071470

§ 371 Date: Mar. 14, 2023

Related U.S. Application Data
(60) Provisional application No. 63/201,599, filed on May 5, 2021.
(60) Provisional application No. 63/079,060, filed on Sep. 16, 2020.

(51) Int. Cl. A61B 5/00; A61B 5/03; A61B 5/107; G06V 10/44; G06V 10/764; G06V 10/82

(52) U.S. Cl. CPC A61B 5/4233; A61B 5/037; A61B 5/1076; G06V 10/44; G06V 10/764; G06V 10/82

(57) ABSTRACT

Measurements of esophageal pressure and geometry are classified using a trained machine learning algorithm, such as a neural network or other classifier algorithm. Contractile response patterns can be identified in the esophageal pressure and geometry data, from which classified feature data can be generated. The classified feature data classify the esophageal pressure and geometry data as being indicative of an upper gastrointestinal disorder in the subject.

### CROSS-REFERENCE TO RELATED APPLICATIONS

This application claims the benefit of U.S. Provisional Patent Application Ser. No. 63/079,060 filed on Sep. 16, 2020, and entitled “CLASSIFICATION OF FUNCTIONAL LUMEN IMAGING PROBE DATA,” and of U.S. Provisional Patent Application Ser. No. 63/201,599 filed on May 5, 2021, and entitled “CLASSIFICATION OF FUNCTIONAL LUMEN IMAGING PROBE DATA,” both of which are herein incorporated by reference in their entirety.

### STATEMENT REGARDING FEDERALLY SPONSORED RESEARCH

N/A

### BACKGROUND

Currently the assessment of motility disorders of the esophagus is focused on using a transnasal catheter to perform pressure assessment while the patient is awake. The functional lumen imaging probe (“FLIP”) was developed to circumvent the problem of having patients do this procedure while they are awake and unsedated. A FLIP utilizes high-resolution impedance planimetry to measure luminal dimensions during controlled, volumetric distension of a balloon-positioned within the esophagus. Esophageal contractility can be elicited by FLIP distension and identified when esophageal diameter changes are depicted as a function of time. FLIP can therefore detect esophageal contractions that both occlude and do not occlude the esophageal lumen (i.e. non-occluding contractions).

Unfortunately, the FLIP technology lacks a validated analysis platform, and diagnosis is made loosely based on pattern recognition and a few numerical measures of distensibility. There remains a need for a tool that can help the clinician diagnose major motor disorders and normal function based on FLIP data.

### SUMMARY OF THE DISCLOSURE

The present disclosure addresses the aforementioned drawbacks by providing a method for generating classified feature data indicative of an upper gastrointestinal disorder in a subject based on esophageal measurement data acquired from the subject's esophagus. The method includes accessing esophageal measurement data with a computer system, where the esophageal measurement data comprise measurements of pressure within the subject's esophagus and changes in a geometry of the subject's esophagus. A trained machine learning algorithm is also accessed with the computer system, where the trained machine learning algorithm has been trained on training data in order to generate classified feature data from esophageal measurement data. The esophageal measurement data are applied to the trained machine learning algorithm using the computer system, generating output as classified feature data that classify the esophageal measurement data as being indicative of an upper gastrointestinal disorder in the subject.

It is another aspect of the present disclosure to provide a method for generating a report that classifies an upper gastrointestinal disorder in a subject. The method includes accessing functional lumen imaging probe (FLIP) data with a computer system, where the FLIP data depict esophageal pressure and diameter measurements in the subject's esophagus. A trained classification algorithm is also accessed with the computer system. Classified feature data are generated with the computer system by inputting the FLIP data to the trained classification algorithm, generating output as the classified feature data, wherein the classified feature data classify the FLIP data as being indicative of an upper gastrointestinal disorder in the subject. A report is then generated from the classified feature data using the computer system, where the report indicates a classification of the FLIP data being indicative of the upper gastrointestinal disorder in the subject.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a block diagram of an example system for classifying esophageal measurement data (e.g., manometry data, panometry data, FLIP data).

FIG. 2 is a block diagram of example components that can implement the system of FIG. 1.

FIG. 3 is a flowchart setting forth the steps of an example method for generating classified feature data, which indicate a classification and/or probability score of an upper gastrointestinal disorder in a subject, by processing esophageal measurement data with an AI-based classifier, which may implement a machine learning based classifier in some instances.

FIG. 4 is a flowchart setting forth the steps of an example method for generating classified feature data, which indicate a classification and/or probability score of an upper gastrointestinal disorder in a subject, by inputting esophageal measurement data to a suitably trained neural network or other machine learning algorithm.

FIG. 5 is a flowchart setting forth the steps of an example method for training a neural network or other machine learning algorithm to generate classified feature data from input esophageal measurement data.

FIGS. 6A-6F show example distention-induced contractility patterns in esophageal measurement data, which can be labeled as labeled data, including a RAC pattern (FIG. 6A), an ACR pattern (FIG. 6B), an RRC pattern (FIG. 6C), a distention induced contractility pattern (FIG. 6D), a repeating pattern of RACs with six contractions per minute (FIG. 6E), and a repeating pattern of RACs with twelve contractions per minute (FIG. 6F).

FIGS. 7A and 7B illustrate examples of contractile response patterns in esophageal measurement data.

FIGS. 8A and 8B shows an example SOC pattern (FIG. 8A) and LES-L pattern (FIG. 8B) in esophageal measurement data.

FIG. 9 shows examples of additional contractile response patterns in esophageal measurement data.

FIG. 10 shows an example scheme for labeling contractile response patterns in esophageal measurement data.

FIG. 11A shows an example table of EGJ-DI values.

FIG. 11B shows an example association of FLIP panometry EGJ opening parameters with EGJ obstruction based on a Chicago Classification v4.0.

FIG. 12A shows an example classification scheme based on EGJ-DI values and contractile response patterns.

FIG. 12B shows an example workflow for classifying an upper gastrointestinal disorder in a subject based on esophageal measurement data using classification schemes described in the present disclosure.

FIG. 13 is another example classification scheme based on EGJ-DI values and contractile response patterns, which implements a convolutional neural network.

FIG. 14 is yet another example classification scheme based on EGJ-DI values and contractile response patterns.

FIG. 15 is still another example classification scheme based on EGJ-DI values and contractile response patterns.

FIG. 16 is an example prediction model for a classification scheme based on EGJ-DI values and contractile response patterns.

FIG. 17 is another example prediction model for a classification scheme based on EGJ-DI values and contractile response patterns.

FIG. 18 is an example classification scheme for an absent contractile response (“ACR”) pattern.

FIG. 19 is an example classification scheme for a spastic contractile response (“SCR”) pattern.

FIG. 20 is an example classification scheme for a borderline/diminished contractile response (“BDCR”) pattern.

FIG. 21 is an example classification scheme for an impaired-disordered contractile response (“IDCR”) pattern.

FIG. 22 is an example of random forest-based classifier models for generating classified feature data according to some embodiments described in the present disclosure.

FIG. 23 is an example classification of esophageal motility based on contractile response patterns and EGJ opening classification.

FIG. 24 is an example association between FLIP panometry findings and Chicago Classification v4.0 (CCv4.0) high-resolution manometry diagnoses.

FIG. 25 illustrates a distribution of CCv4.0 diagnoses among example FLIP panometry motility classifications.

### DETAILED DESCRIPTION

Described here are systems and methods for classifying upper gastrointestinal (“UGI”) data, which may include manometry data, panometry data, and/or other data acquired from a subject's UGI tract or a portion thereof (e.g., the subject's esophagus) using, for example, a functional lumen imaging probe (“FLIP”) or other measurement device. The systems and methods described in the present disclosure implement classification algorithms, machine learning algorithms, or combinations thereof, in order to classify these data. For instance, patterns in the input data can be identified and classified using one or more classification and/or machine learning algorithms.

In general, the systems and methods described in the present disclosure provide an artificial intelligence (“AI”) methodology to classify esophageal measurement data into relevant pathologic groups, including esophageal measurement data acquired from functional lumen imaging for esophageal function testing. In some embodiments, the classification may be a binary classification, in which the esophageal measurement data are classified into one of two categories or class labels (e.g., “normal” and “abnormal”). In these instances, classification algorithms including logistic regression, k-nearest neighbors, decision trees, support vector machines, Naive Bayes, and/or artificial neural networks can be implemented.

In some other embodiments, the classification may be a multiclass classification, in which the esophageal measurement data are classified into more than two categories or class labels (e.g., “normal,” “abnormal-not achalasia,” and “abnormal-achalasia”). In these instances, classification algorithms including k-nearest neighbors, decision trees, Naive Bayes, random forest, gradient boosting, and/or artificial neural networks (e.g., convolutional neural networks) can be implemented.

In still other embodiments, the classification may be a multilabel classification, in which the esophageal measurement data are classified into two or more categories or class labels, and where two or more class labels can be predicted for each data sample. For example, a data sample may be classified as “normal” or “abnormal” and an “abnormal” class may be additionally classified as “not achalasia” or “achalasia.” In these instances, classification algorithms including multi-label decision trees, multi-label random forests, multi-label gradient boosting, and/or artificial neural networks (e.g., convolutional neural networks) can be implemented.

In one example, a neural network, such as a convolutional neural network, that is focused on heat maps estimated, computed, or otherwise determined from esophageal measurement data can be used to classify the esophageal measurement data into one of three distinct patterns: normal, abnormal-not achalasia, and abnormal-achalasia. Classifying patients into one of these three groups can help inform a clinician's decision for treatment and management.

The following acronyms, used throughout the present disclosure, have the associated definition given in the table below, although other acronyms may be introduced in the detailed description:

TABLE 1
Acronyms

| | |
|---|---|
| ABNL | abnormal |
| AC | antegrade contraction |
| ACH | achalasia |
| ACR | absent contractile response |
| AI | artificial intelligence |
| BCR | borderline contractile response |
| BDCR | borderline/diminished contractile response |
| BEO | borderline EGJ opening |
| BnEO | borderline normal EGJ opening |
| BrEO | borderline reduced EGJ opening |
| CBT | cognitive-behavioral therapy |
| CNN | convolutional neural network |
| CVD | cardiovascular disease |
| DES | diffuse esophageal spasm |
| DP | defective peristalsis |
| EGD | esophagogastroduodenoscopy |
| EGJ | esophagogastric junction |
| EGJ-DI | EGJ distensibility index |
| EGJOO | EGJ outflow obstruction |
| EoE | eosinophilic esophagitis |
| FLIP | functional lumen imaging probe |
| FPEGJOO | fragmented peristalsis and EGJOO |
| GDH | glutamate dehydrogenase |
| GERD | gastroesophageal reflux disease |
| HE | hypercontractile esophagus |
| HRM | high-resolution manometry |
| IBP | intra-bolus pressure |
| IDCR | impaired-disordered contractile response |
| IEM | ineffective esophageal motility |
| IRP | integrated relaxation pressure |
| JH | jackhammer |
| LES | lower esophageal sphincter |
| LES-L | LES lift |
| MMCD | median mid-contractile diameter |
| MMD | mass median diameter |
| NCR | normal contractile response |
| NEO | normal EGJ opening |
| NL | normal |
| NPV | negative predictive value |
| PD | pneumatic dilation |
| POEM | peroral endoscopic myotomy |
| PPV | positive predictive value |
| RAC | repetitive antegrade contraction |
| RC | retrograde contraction |
| REO | reduced EGJ opening |
| RO6 | rule of sixes |
| RRC | repetitive retrograde contraction |
| SCR | spastic contractile response |
| sLESC | sustained LES contraction |
| SOC | sustained occluding contractions |
| SRCR | spastic-reactive contractile response |
| SSC | systemic sclerosis |
| TBE | timed barium esophagram |
| UGI | upper gastrointestinal |

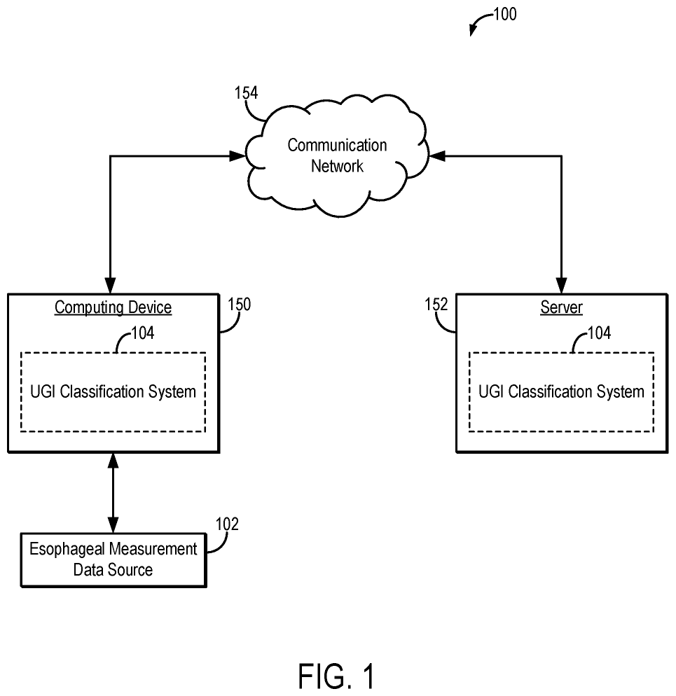

Referring now to FIG. 1, an example of a system 100 for classifying esophageal measurement data (e.g., manometry data, panometry data, and/or other FLIP data) or other UGI measurement data in accordance with some embodiments of the systems and methods described in the present disclosure is shown. In some embodiments, the esophageal measurement data may include esophageal measurement data acquired from a subject's esophagus, and may include manometry data, panometry data, and/or FLIP data. As shown in FIG. 1, a computing device 150 can receive one or more types of esophageal measurement data (e.g., manometry data, panometry data, FLIP data) from esophageal measurement data source 102. In some embodiments, computing device 150 can execute at least a portion of an UGI classification system 104 to classify esophageal measurement data (e.g., manometry data, panometry data, FLIP data, which may be acquired from a subject's esophagus or other portion of the subject's UGI tract) received from the esophageal measurement data source 102 and/or to generate feature data or maps based on the esophageal measurement data received from the esophageal measurement data source 102. For instance, feature data and/or feature maps may indicate a probability of a pathology, functional state of the UGI tract or portion thereof (e.g., the esophagus), or other diagnosis; a class or class label corresponding to a pathology, functional state of the UGI tract or portion thereof (e.g., the esophagus), or other diagnosis; and the like.

Additionally or alternatively, in some embodiments, the computing device 150 can communicate information about data received from the esophageal measurement data source 102 to a server 152 over a communication network 154, which can execute at least a portion of the UGI classification system 104. In such embodiments, the server 152 can return information to the computing device 150 (and/or any other suitable computing device) indicative of an output of the UGI classification system 104.

In some embodiments, computing device 150 and/or server 152 can be any suitable computing device or combination of devices, such as a desktop computer, a laptop computer, a smartphone, a tablet computer, a wearable computer, a server computer, a virtual machine being executed by a physical computing device, and so on.

In some embodiments, esophageal measurement data source 102 can be any suitable source of data (e.g., measurement data, manometry data, panometry data, FLIP data, images or maps reconstructed from such data), such as a functional lumen imaging probe or other suitable imaging or functional measurement device, another computing device (e.g., a server storing data), and so on. In some embodiments, esophageal measurement data source 102 can be local to computing device 150. For example, esophageal measurement data source 102 can be incorporated with computing device 150 (e.g., computing device 150 can be configured as part of a device for capturing, scanning, and/or storing data). As another example, esophageal measurement data source 102 can be connected to computing device 150 by a cable, a direct wireless link, and so on. Additionally or alternatively, in some embodiments, esophageal measurement data source 102 can be located locally and/or remotely from computing device 150, and can communicate data to computing device 150 (and/or server 152) via a communication network (e.g., communication network 154).

In some embodiments, communication network 154 can be any suitable communication network or combination of communication networks. For example, communication network 154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 154 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, and so on.

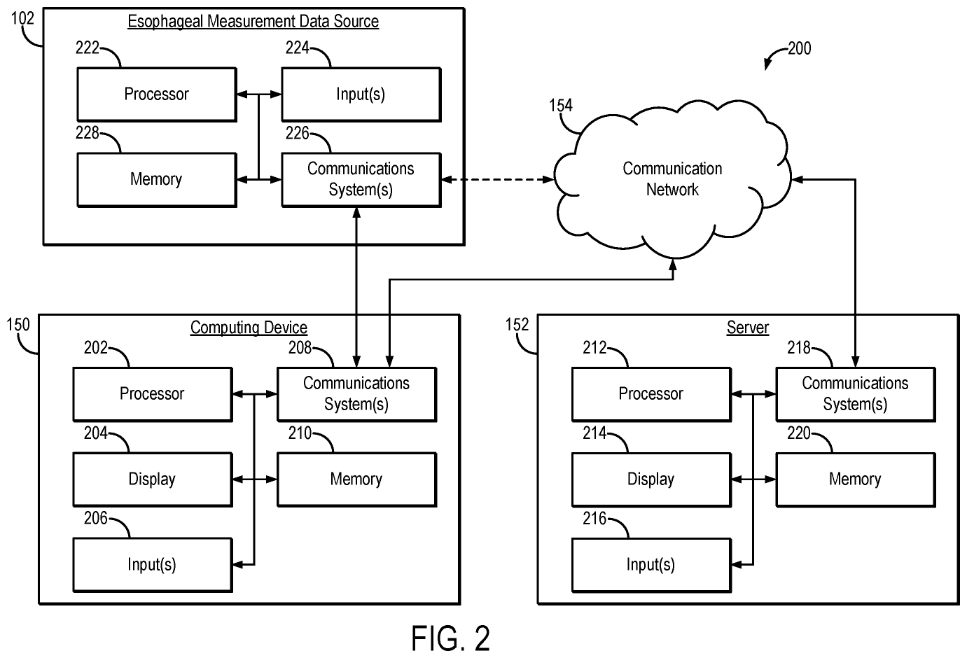

Referring now to FIG. 2, an example of hardware 200 that can be used to implement esophageal measurement data source 102, computing device 150, and server 152 in accordance with some embodiments of the systems and methods described in the present disclosure is shown. As shown in FIG. 2, in some embodiments, computing device 150 can include a processor 202, a display 204, one or more inputs 206, one or more communication systems 208, and/or memory 210. In some embodiments, processor 202 can be any suitable hardware processor or combination of processors, such as a central processing unit (“CPU”), a graphics processing unit (“GPU”), and so on. In some embodiments, display 204 can include any suitable display devices, such as a computer monitor, a touchscreen, a television, and so on. In some embodiments, inputs 206 can include any suitable input devices and/or sensors that can be used to receive user input, such as a keyboard, a mouse, a touchscreen, a microphone, and so on.

In some embodiments, communications systems 208 can include any suitable hardware, firmware, and/or software for communicating information over communication network 154 and/or any other suitable communication networks. For example, communications systems 208 can include one or more transceivers, one or more communication chips and/or chip sets, and so on. In a more particular example, communications systems 208 can include hardware, firmware and/or software that can be used to establish a Wi-Fi connection, a Bluetooth connection, a cellular connection, an Ethernet connection, and so on.

In some embodiments, memory 210 can include any suitable storage device or devices that can be used to store instructions, values, data, or the like, that can be used, for example, by processor 202 to present content using display 204, to communicate with server 152 via communications system(s) 208, and so on. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, and so on. In some embodiments, memory 210 can have encoded thereon, or otherwise stored therein, a computer program for controlling operation of computing device 150. In such embodiments, processor 202 can execute at least a portion of the computer program to present content (e.g., images, heat maps, user interfaces, graphics, tables), receive content from server 152, transmit information to server 152, and so on.

In some embodiments, server 152 can include a processor 212, a display 214, one or more inputs 216, one or more communications systems 218, and/or memory 220. In some embodiments, processor 212 can be any suitable hardware processor or combination of processors, such as a CPU, a GPU, and so on. In some embodiments, display 214 can include any suitable display devices, such as a computer monitor, a touchscreen, a television, and so on. In some embodiments, inputs 216 can include any suitable input devices and/or sensors that can be used to receive user input, such as a keyboard, a mouse, a touchscreen, a microphone, and so on.

In some embodiments, communications systems 218 can include any suitable hardware, firmware, and/or software for communicating information over communication network 154 and/or any other suitable communication networks. For example, communications systems 218 can include one or more transceivers, one or more communication chips and/or chip sets, and so on. In a more particular example, communications systems 218 can include hardware, firmware and/or software that can be used to establish a Wi-Fi connection, a Bluetooth connection, a cellular connection, an Ethernet connection, and so on.

In some embodiments, memory 220 can include any suitable storage device or devices that can be used to store instructions, values, data, or the like, that can be used, for example, by processor 212 to present content using display 214, to communicate with one or more computing devices 150, and so on. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, and so on. In some embodiments, memory 220 can have encoded thereon a server program for controlling operation of server 152. In such embodiments, processor 212 can execute at least a portion of the server program to transmit information and/or content (e.g., data, images, a user interface) to one or more computing devices 150, receive information and/or content from one or more computing devices 150, receive instructions from one or more devices (e.g., a personal computer, a laptop computer, a tablet computer, a smartphone), and so on.

In some embodiments, esophageal measurement data source 102 can include a processor 222, one or more inputs 224, one or more communications systems 226, and/or memory 228. In some embodiments, processor 222 can be any suitable hardware processor or combination of processors, such as a CPU, a GPU, and so on. In some embodiments, the one or more inputs 224 are generally configured to acquire data and can include a functional lumen imaging probe. Additionally or alternatively, in some embodiments, one or more inputs 224 can include any suitable hardware, firmware, and/or software for coupling to and/or controlling operations of a functional lumen imaging probe. In some embodiments, one or more portions of the one or more inputs 224 can be removable and/or replaceable.

Note that, although not shown, esophageal measurement data source 102 can include any suitable inputs and/or outputs. For example, esophageal measurement data source 102 can include input devices and/or sensors that can be used to receive user input, such as a keyboard, a mouse, a touchscreen, a microphone, a trackpad, a trackball, and so on. As another example, esophageal measurement data source 102 can include any suitable display devices, such as a computer monitor, a touchscreen, a television, etc., one or more speakers, and so on.

In some embodiments, communications systems 226 can include any suitable hardware, firmware, and/or software for communicating information to computing device 150 (and, in some embodiments, over communication network 154 and/or any other suitable communication networks). For example, communications systems 226 can include one or more transceivers, one or more communication chips and/or chip sets, and so on. In a more particular example, communications systems 226 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 228 can include any suitable storage device or devices that can be used to store instructions, values, data, or the like, that can be used, for example, by processor 222 to control the one or more inputs 224; to receive data from the one or more inputs 224; to generate images, heat maps, and/or computed parameters from data; to present content (e.g., images, heat maps, a user interface) using a display; to communicate with one or more computing devices 150; and so on. Memory 228 can include any suitable volatile memory, non-volatile memory, storage, or any suitable combination thereof. For example, memory 228 can include RAM, ROM, EEPROM, one or more flash drives, one or more hard disks, one or more solid state drives, one or more optical drives, and so on. In some embodiments, memory 228 can have encoded thereon, or otherwise stored therein, a program for controlling operation of esophageal measurement data source 102. In such embodiments, processor 222 can execute at least a portion of the program to compute parameters, transmit information and/or content (e.g., data, images, heat maps) to one or more computing devices 150, receive information and/or content from one or more computing devices 150, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

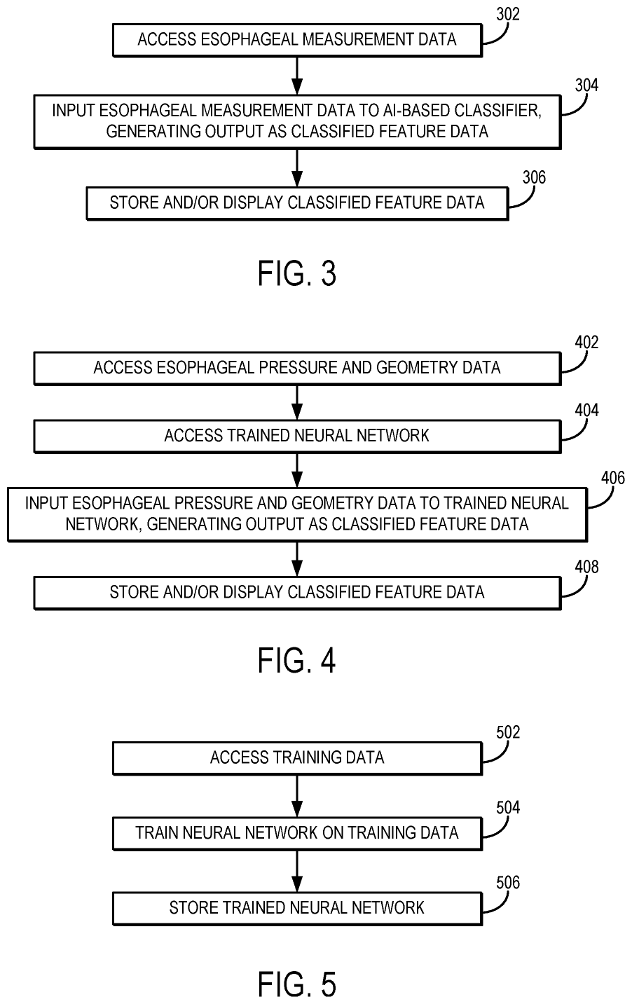

Referring now to FIG. 3, a flowchart is illustrated as setting forth the steps of an example method for generating classified feature data based on measurement data obtained from a subject's upper gastrointestinal tract, such as the subject's esophagus, where the classified feature data are indicative of a classification and/or probability score of an upper gastrointestinal disorder, or other class label of the measurement data, in the subject.

The method includes accessing esophageal measurement data or other UGI measurement data with a computer system, as indicated at step 302. For instance, the computing device 150 (or the server 152) can access the esophageal measurement data from the esophageal measurement data source 102 through either a wired connection or a wireless connection, as described above. In some embodiments, the esophageal measurement data can include measurement data indicating measurements of one or more characteristics of the UGI tract, such as pressure and/or geometry (e.g., lumen diameter or other geometric measurements). For example, the esophageal measurement data can include measurements of pressure and/or geometry of the subject's UGI tract or a portion thereof (e.g., the esophagus).

As one non-limiting example, esophageal measurement data are esophageal measurement data that indicate measurements of pressure and/or geometry of the subject's esophagus. The esophageal pressure and geometry data can be FLIP data acquired from the subject's esophagus using a FLIP system, and may include in a non-limiting example, measurement data such as pump status (e.g., inflated, deflated, or stopped), readings from the sensor pairs on the catheter balloon that indicate the diameter of the lumen, balloon pressure, balloon volume, and/or balloon temperature. Additionally or alternatively, the esophageal pressure and geometry data may include other manometry, planimetry, and/or panometry data. The esophageal pressure and geometry data may include measurement values or plots or measurement values. In some instances, the esophageal pressure and geometry data may include two-dimensional images, or heat maps, that depict a spatial and/or spatiotemporal distribution of esophageal pressure and/or geometric measurement values.

Accessing the esophageal measurement data may include retrieving such data from a memory or other suitable data storage device or medium. Alternatively, accessing the input data may include acquiring such data with a suitable measurement device, such as a functional lumen imaging probe, and transferring or otherwise communicating the data to the computer system.

In some instances, the esophageal measurement data can include measurements of esophageal pressure and/or geometry that may include artifacts, such as artifacts related to the diameter measured during periods of strong esophageal contraction. During contractions where the lumen is occluded, the measurements may be negated as the contraction can interrupt the flow of current within the catheter. These artifacts can therefore be detected in the data, and the data processed accordingly to remove the artifacts.

The esophageal measurement data are then input to an AI-based classifier, generating output as classified feature data, as indicated at step 304. For instance, the processor 202 of the computing device 150 (or the processor 212 of the server 152) receives the esophageal measurement data and provides the esophageal measurement data as input data to an AI-based classifier executed by the processor 202 (or processor 212), generating output data as the classified feature data. The AI-based classifier can be implemented by the processor 202 executing an AI classifier program, algorithm, or model stored in the memory 210 of the computer device 150, or alternatively by the processor 212 executing an AI classifier program, algorithm, or model stored in the memory 220 of the server 152. For example, the AI classifier program, algorithm, or model executing on the processor 202 (or processor 212) processes (e.g., classifies according to one of the machine learning and/or artificial intelligence algorithms described in the present disclosure) the received esophageal measurement data and generates an output as the classified feature data.

The classified feature data may include a classification of the subject as belonging to a particular classification of upper gastrointestinal disorder, a quantifiable probability score of the subject belonging to one or more upper gastrointestinal disorders, and the like. As one example, the classified feature data may indicate the probability for a particular classification (i.e., the probability that a subject belongs to a particular class), such as normal, abnormal-not achalasia, and abnormal-achalasia.

In some embodiments, the computing device 150 and/or server 152 may store a selection of various AI-based classifiers, in which each AI-based classifier is specifically configured to perform a different classification task. In such embodiments, the user may select which of the AI-based classifiers to implement with the computing device 150 and/or server 152. For example, the computing device 150 or another external device (e.g., a smartphone, a tablet computer, a cellular phone, a laptop computer, a smart watch, and the like) may provide a graphical interface that allows the user to select a type of AI-based classifier. A user may select the AI-based classifier based on, for example, the type of esophageal measurement data available for the subject.

As described above, the AI-based classifier may implement any number of suitable AI classification programs, algorithms, and/or models, including logistic regression, k-nearest neighbors, decision trees, support vector machines, Naive Bayes, random forest, gradient boosting, and/or artificial neural networks (e.g., convolutional neural networks).

In some embodiments, more than one AI-based classifier can be implemented to process the esophageal measurement data. For example, esophageal measurement data can be input to a first AI-based classifier to generate output as first classified feature data. The esophageal measurement data, first classified feature data, or both, can then be input to a second AI-based classifier to generate output as second classified feature data. The first classified feature data may indicate the presence of one or more contractile patterns in the esophageal measurement data, as an example. The presence and/or identification of these contractile patterns can be used as an input to a second AI-based classifier, in addition to other esophageal measurement data or other data (e.g., parameters that are computed or estimated from esophageal measurement data). The second classified feature data can then indicate a classification of the esophageal measurement data as indicating a particular condition, such as a normal condition, an abnormal but inconclusive for achalasia condition, or an achalasia condition.

The classified feature data generated by processing the esophageal measurement data using the processor 202 and/or processor 212 executing an AI-based classifier can then be displayed to a user, stored for later use or further processing, or both, as indicated at step 306. For example, the classified feature data may be stored locally by the computer device 150 (e.g., in the memory 210) or displayed to the user via the display 204 of the computing device 150. Additionally or alternatively, the classified feature data may be stored in the memory 220 of the server 152 and/or displayed to a user via the display 214 of the server 152. In still other embodiments, the classified feature data may be stored in a memory or other data storage device or medium other than those associated with the computing device 150 or server 152. In these instances, the classified feature data can be transmitted to such other devices using the communication network 154 or other wired or wireless communication links.

In one example, the computer system (e.g., computing device 150, server 152) implements an artificial neural network for the AI-based classifier. The artificial neural network generally includes an input layer, one or more hidden layers or nodes, and an output layer. Typically, the input layer includes as many nodes as inputs provided to the computer system. As described above, the number (and the type) of inputs provided to the computer system may vary based on the particular task for the AI-based classifier. Accordingly, the input layer of the artificial neural network may have a different number of nodes based on the particular task for the AI-based classifier.

In some embodiments, the input to the AI-based classifier may include esophageal measurement data such as pump status (e.g., inflated, deflated, or stopped), readings from the sensor pairs on the catheter balloon that indicate the diameter of the lumen, balloon pressure, balloon volume, and/or balloon temperature, which may be measured with a FLIP system or other suitable measurement system or device.

The input layer connects to the one or more hidden layers. The number of hidden layers varies and may depend on the particular task for the AI-based classifier. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. However, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is associated with an activation function. The activation function defines how the hidden layer is to process the input received from the input layer or from a previous input or hidden layer. These activation functions may vary and be based on not only the type of task associated with the AI-based classifier, but may also vary based on the specific type of hidden layer implemented.

Each hidden layer may perform a different function. For example, some hidden layers can be convolutional hidden layers which can, in some instances, reduce the dimensionality of the inputs, while other hidden layers can perform more statistical functions such as max pooling, which may reduce a group of inputs to the maximum value, an averaging layer, among others. In some of the hidden layers, each node may be connected to each node of the next hidden layer. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the AI-based classifier is a multiclass classifier, the output layer may include, for example, a number of different nodes, where each different node corresponds to a different class or label of the esophageal measurement data. A first node may indicate that the esophageal measurement data are classified as a normal class type, a second node may indicate that the esophageal measurement data are classified as an abnormal-not achalasia class type, and a third node may indicate that the esophageal measurement data are classified as an abnormal-achalasia class type. Additionally or alternatively, an additional node may indicate that the esophageal measurement data corresponds to an unknown (or unidentifiable) class. In some embodiments, the computer system then selects the output node with the highest value and indicates to the computer system or to the user the corresponding classification of the esophageal measurement data (e.g., by outputting and/or displaying the classified feature data). In some embodiments, the computer system may also select more than one output node.

Referring now to FIG. 4, a flowchart is illustrated as setting forth the steps of an example method for generating classified feature data using a suitably trained neural network or other machine learning algorithm, where the classified feature data are indicative of a classification and/or probability score of an upper gastrointestinal disorder in a subject.

The method includes accessing esophageal measurement data, which may include esophageal pressure and geometry (e.g., diameter or other geometric measurements) data with a computer system, as indicated at step 402. As one non-limiting example, the esophageal pressure and geometry data can be FLIP data acquired from a subject's esophagus using a FLIP system. Additionally or alternatively, the esophageal pressure and geometry data may include other manometry, planimetry, and/or panometry data. The esophageal pressure and geometry data may include measurement values or plots or measurement values. In some instances, the esophageal pressure and geometry data may include two-dimensional images, or heat maps, that depict a spatial and/or spatiotemporal distribution of esophageal pressure and/or geometric measurement values. Additionally or alternatively, the esophageal measurement data may include data such as pump status (e.g., inflated, deflated, or stopped), readings from the sensor pairs on the catheter balloon that indicate the diameter of the lumen, balloon pressure, balloon volume, and/or balloon temperature.

Accessing the esophageal measurement data may include retrieving such data from a memory or other suitable data storage device or medium. Alternatively, accessing the esophageal measurement data may include acquiring such data with a suitable measurement device, such as a functional lumen imaging probe, and transferring or otherwise communicating the data to the computer system.

In some instances, the measurements of esophageal pressure and/or geometry may include artifacts, such as artifacts related to the diameter measured during periods of strong contraction. During contractions where the lumen is occluded, the measurements may be negated as the contraction can interrupt the flow of current within the catheter. These artifacts can therefore be detected in the data, and the data processed accordingly to remove the artifacts.

A trained neural network (or other suitable machine learning algorithm) is then accessed with the computer system, as indicated at step 404. Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed. As a non-limiting example, the trained neural network may be a trained convolutional neural network.

In general, the neural network is trained, or has been trained, on training data in order to identify patterns (e.g., contractile response patterns) in the esophageal pressure and geometry data, classify the esophageal pressure and geometry data based on the identified patterns, and to generate output as classified data and/or feature data representative of different upper gastrointestinal disorder classifications and/or probability scores of different upper gastrointestinal disorder classifications.

The esophageal pressure and geometry data are then input to the trained neural network, generating output as classified feature data, as indicated at step 406. For example, the classified feature data may include a classification of the subject as belonging to a particular classification of upper gastrointestinal disorder, a quantifiable probability score of the subject belonging to one or more upper gastrointestinal disorders, and the like. As one example, the classified feature data may indicate the probability for a particular classification (i.e., the probability that a subject belongs to a particular class), such as normal, abnormal-not achalasia, and abnormal-achalasia.

In some embodiments, the classified feature data may indicate that a particular distention-induced contractility pattern is present in the esophageal measurement data. Examples of different distention-induced contractility patterns are described below with respect to the labeling of training data (e.g., with respect to FIG. 5). The identification of one or more distention-induced contractility patterns can be provided as classified feature data in addition to other types of classified feature data described in the present disclosure. For example, the classified feature data may indicate that the esophageal measurement data are classified as an “abnormal-not achalasia” class, and also that certain distention-induced contractility patterns were identified in the esophageal measurement data. As such, a clinician may evaluate both the classification of the esophageal measurement data and the identified distention-induced contractility patterns to assist in making a diagnosis for the subject.

The classified feature data generated by inputting the esophageal measurement data to the trained neural network(s) can then be displayed to a user, stored for later use or further processing, or both, as indicated at step 408.

Referring now to FIG. 5, a flowchart is illustrated as setting forth the steps of an example method for training one or more neural networks (or other suitable machine learning algorithms) on training data, such that the one or more neural networks are trained to receive input as esophageal measurement data (or other esophageal measurement data) in order to generate output as classified feature data that indicate a classification of the subject as belonging to a particular classification of upper gastrointestinal disorder, a quantifiable probability score of the subject belonging to one or more upper gastrointestinal disorders, and so on.

In general, the neural network(s) can implement any number of different neural network architectures. For instance, the neural network(s) could implement a convolutional neural network, a residual neural network, or the like. In some instances, the neural network(s) may implement deep learning.

Alternatively, the neural network(s) could be replaced with other suitable machine learning algorithms, such as those based on supervised learning, unsupervised learning, deep learning, ensemble learning, dimensionality reduction, and so on.

The method includes accessing training data with a computer system, as indicated at step 502. Accessing the training data may include retrieving such data from a memory or other suitable data storage device or medium. Alternatively, accessing the training data may include acquiring such data with a FLIP system, or other suitable measurement system, and transferring or otherwise communicating the data to the computer system, which may be a part of the FLIP or other suitable measurement system. In general, the training data can include esophageal measurement data, such as esophageal pressure and diameter measurement data.

Additionally or alternatively, the method can include assembling training data from esophageal measurement data using a computer system. This step may include assembling the esophageal measurement data into an appropriate data structure on which the machine learning algorithm can be trained. Assembling the training data may include assembling esophageal measurement data, segmented esophageal measurement data, labeled esophageal measurement data, and other relevant data. For instance, assembling the training data may include generating labeled data and including the labeled data in the training data. Labeled data may include esophageal measurement data, segmented esophageal measurement data, or other relevant data that have been labeled as belonging to, or otherwise being associated with, one or more different classifications or categories.

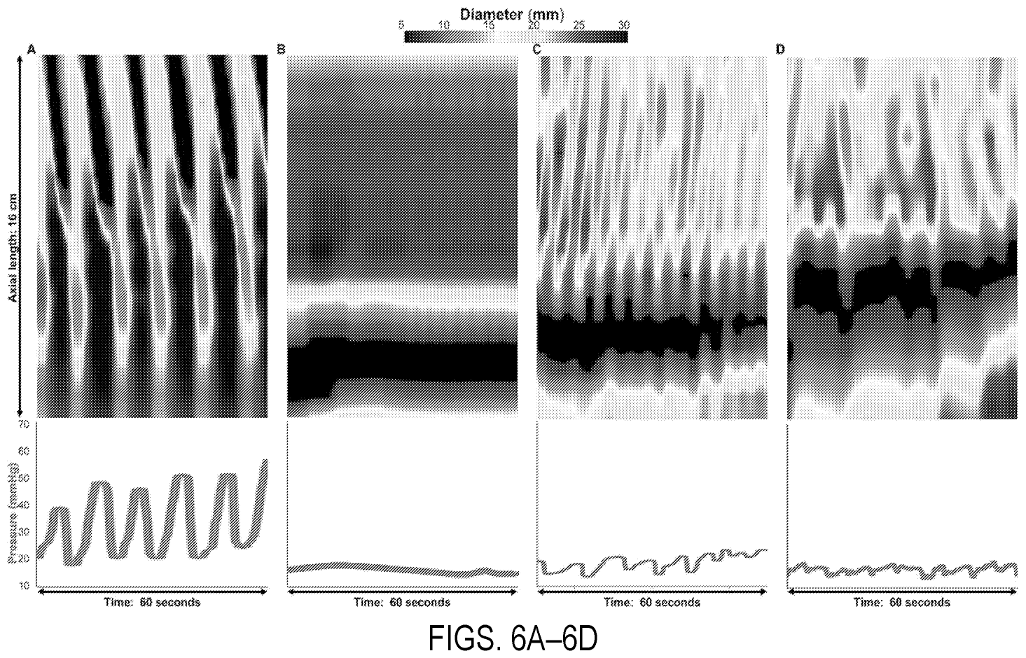

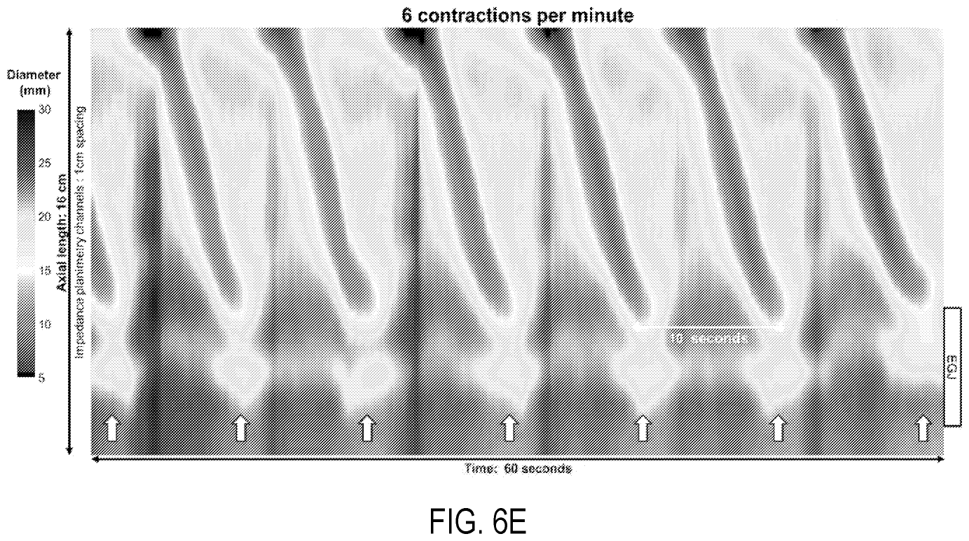

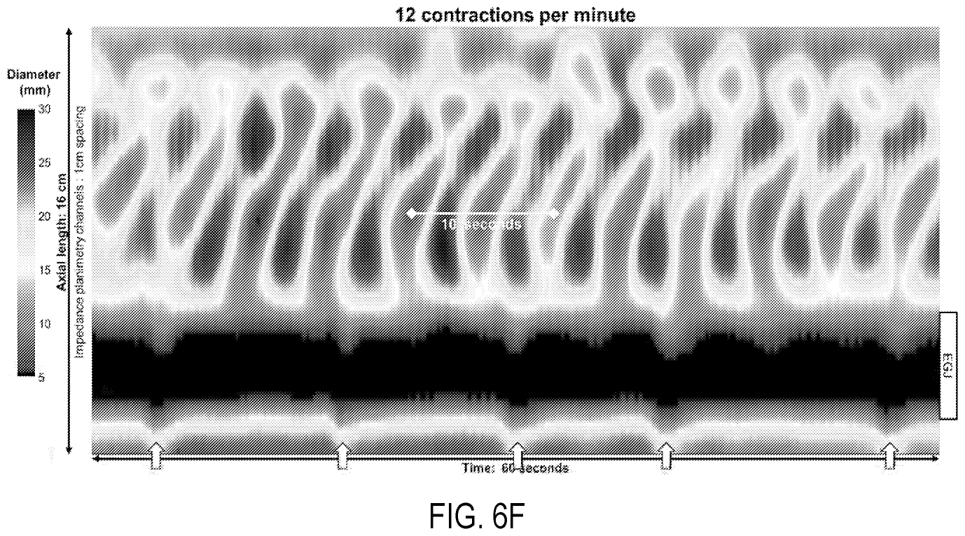

As one non-limiting example, labeled data may include esophageal measurement data and/or segmented esophageal measurement data that have been labeled based on different distention-induced contractility patterns. For instance, the labeled data may include esophageal measurement data labeled as including a repetitive antegrade contractions (“RAC”) pattern, such as the RAC pattern illustrated in FIG. 6A. As another example, the labeled data may include esophageal measurement data labeled as including an absent contractile response (“ACR”), such as the example show in FIG. 6B. Additionally or alternatively, the labeled data can include esophageal measurement data labeled as including repetitive retrograde contractions (“RRCs”), such as illustrated in FIG. 6C. As still another example, the labeled data can include esophageal measurement data labeled as containing distension-induced contractility otherwise not belonging to an identified distinct pattern, such as shown in FIG. 6D.

In some instances, the labeled data may include esophageal measurement data labeled as including a repeating contractile response pattern. As an example, the repeating contractile pattern may include a repeating RAC pattern, such as the repeating RAC patterns shown in FIGS. 6E and 6F. In FIG. 6E, the repeating pattern of RACs includes at least six repeating lumen occlusions longer than 6 cm at a consistent rate of 6±3 per minute. FIG. 6F shows an example repeating pattern of 12 contractions per minute.

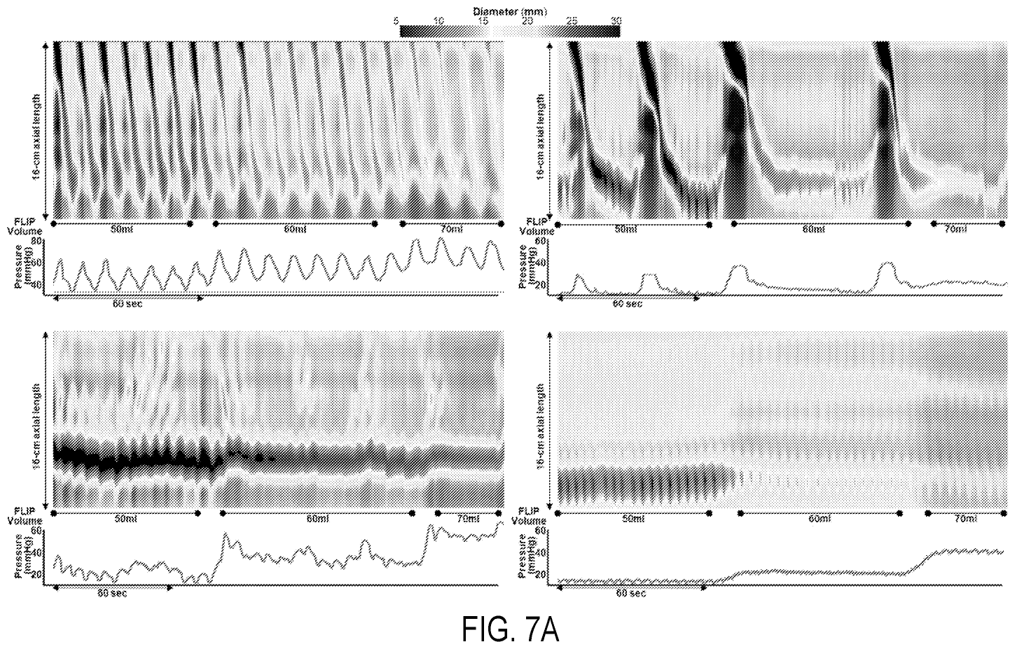

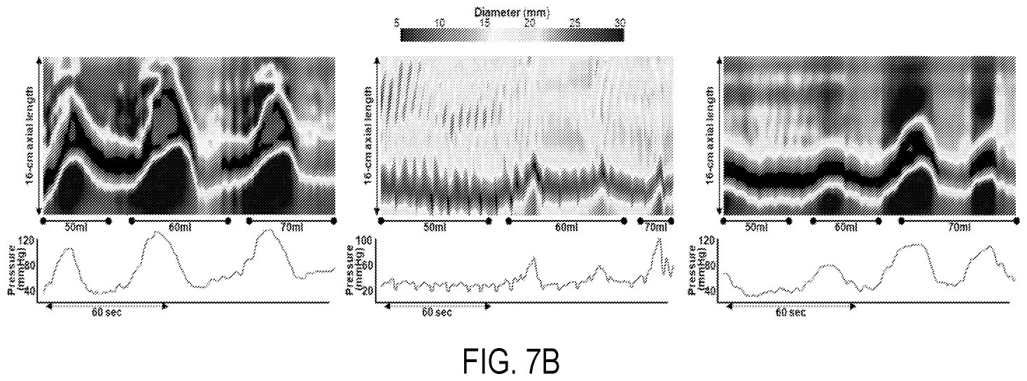

Other example contractile response patterns may include normal contractile response (“NCR”), borderline/diminished contractile response (“BDCR”), borderline contractile response (“BCR”), impaired/disordered contractile response (“ID CR”), spastic contractile response (“SCR”), and/or spastic-reactive contractile response (“SRCR”). Example pathophysiology characterizations and definitions of these contractile response patterns are described below. Examples of these contractile response patterns are illustrated in FIGS. 7A and 7B.

NCR can be representative of a pathophysiology indicating normal neurogenic control and muscular function. As an example, NCR can be defined based on a rule of sixes (“RO6”), in which six normal contractions are observed or otherwise recorded over a period of time, such as per minute. For instance, a RO6 criterion can be satisfied when ≥6 consecutive ACs that are ≥6 cm in axial length occurring at 6±3 AC per minute regular rate.

BCR can be defined as a contractile pattern that does not satisfy the RO6 criterion, in which a distinct AC of at least 6 cm axial length is present, that may have RCs, but not RRCs; and has no SOCs or sLESCs.

BDCR can be representative of a pathophysiology indicating early transition/borderline loss of neurogenic control, which can be evidenced by fewer ACs, delayed triggering at higher volumes, and possible a higher rate of ACs. Additionally or alternatively, BDCR can be representative of a pathophysiology indicating early transition/borderline muscular dysfunction, which can be evidenced by fewer ACs becoming weaker, and may see slower more pronounced contractions that may reflect hypertrophy as an early phase of response to obstruction. As an example, BDCR can be defined as contractile patterns not meeting the RO6 criterion and in which antegrade contractions (“ACs”) are present; retrograde contractions (“RCs”) may be present, but not RRCs; and no sustained occluding contractions (“SOCs”) are present.

IDCR can be representative of a pathophysiology indicating late progression/severe loss of neurogenic control and/or muscular function, which can be evidenced by sporadic or chaotic contractions with no propagation or progressing achalasia, and/or response to distension is not distinct or associated with volume trigger. As an example, IDCR can be defined as contractile patterns in which no distinct ACs are present; that may have sporadic or chaotic contractions not meeting ACs; that may have RCs, but not RRCs; and in which no SOCs are present.

ACR can be representative of a pathophysiology indicating complete loss of neurogenic trigger for secondary peristalsis, which can be related to neuropathy, CVD, diabetes, age, and/or chronic GERD, and may be evidenced by impaired triggering due to dilatation of the wall or loss of compliance. Additionally or alternatively, ACR can be representative of a pathophysiology indicating end-stage muscular dysfunction, such as esophageal dilatation, distortion of the anatomy, and/or atrophy. As an example, ACR can be defined as contractile patterns in which no contractile activity is present (e.g., no contractile activity in the esophageal cavity). In these instances LES-L may be present with no evidence of contraction in the body. As an example, the esophageal measurement data may indicate bag pressures greater than 40 mmHg.

SCR can be representative of a pathophysiology indicating neurogenic disruption leading to reduced latency and sustained contraction, which may be representative of an intrinsic neurogenic dysfunction and/or a response to obstruction. As an example, SCR can be defined as contractile patterns in which SOCs are present, which may have sporadic ACs, and in which RRCs are present (e.g., at least 6 RCs at a rate >9 RCs per minute). Similarly, SRCR can be defined as contractile patterns in which SOCs, sLESCs, or RRCs (at least 6 RCs at a rate >9 RCs per minute) are present, and that may have sporadic ACs.

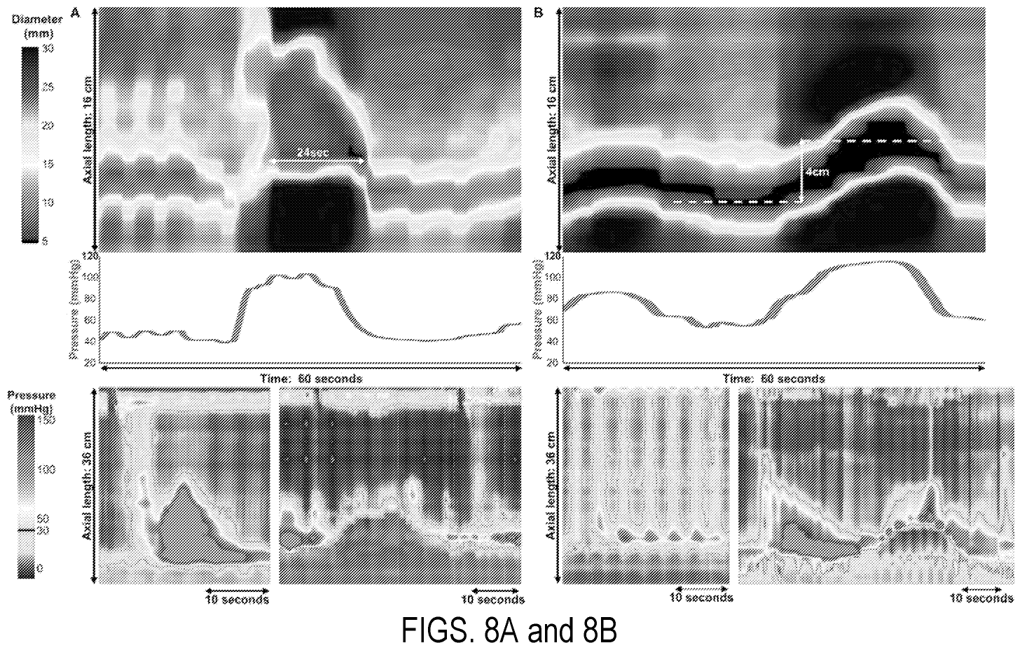

As still another example, the labeled data may include esophageal measurement data that are labeled as containing sustained occluding contractions (“SOCs”), as shown in FIG. 8A. Such patterns may occur in subjects with type III achalasia, and may result in large increases in intra-balloon pressure and an esophageal shortening event with LES-lift (“LES-L”). As shown in FIG. 8B, the labeled data may include esophageal measurement data that are labeled as containing a LES-L. Such patterns may occur in subjects with type II achalasia, and may also be associated with increases in intra-balloon pressure.

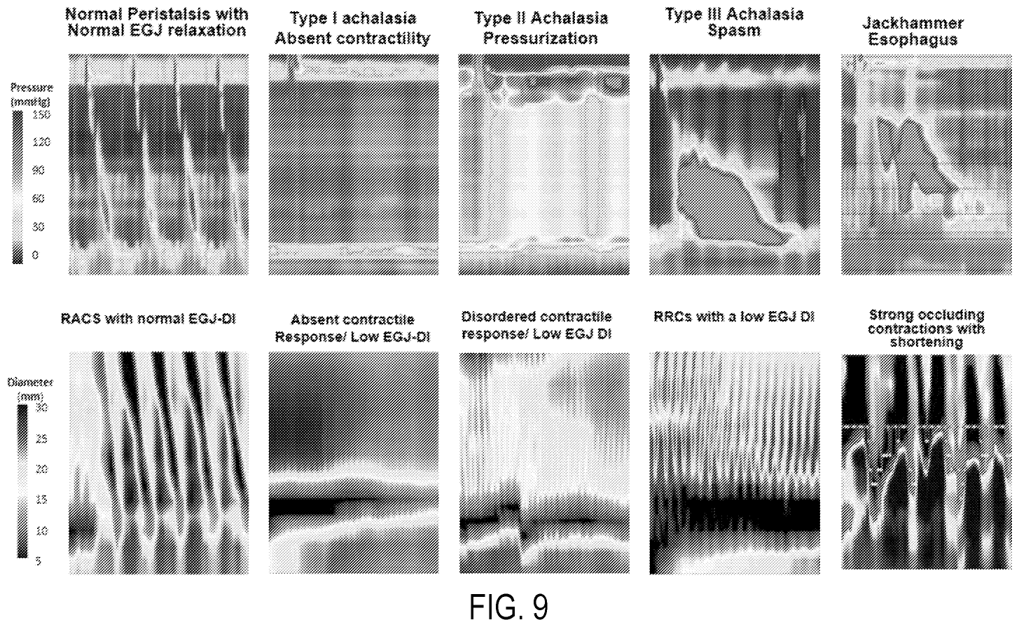

Additional examples of contractile response patterns that can be used when generating labeled data, or which can be identified as classified feature data, are shown in FIG. 9.

As described above, in some instances, the measurements of esophageal pressure and/or geometry may include artifacts, such as artifacts related to the diameter measured during periods of strong contraction. These artifacts can be detected and removed from the esophageal measurement data, as described above.

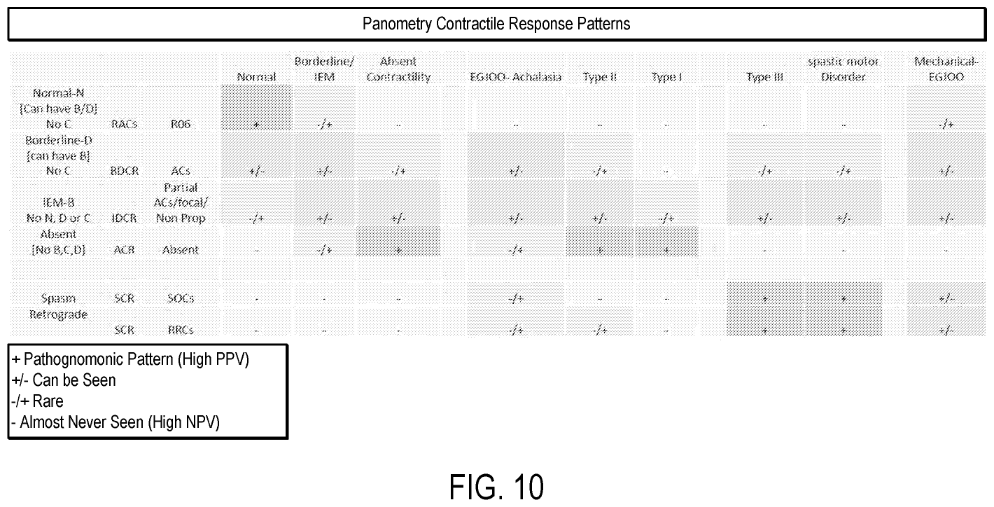

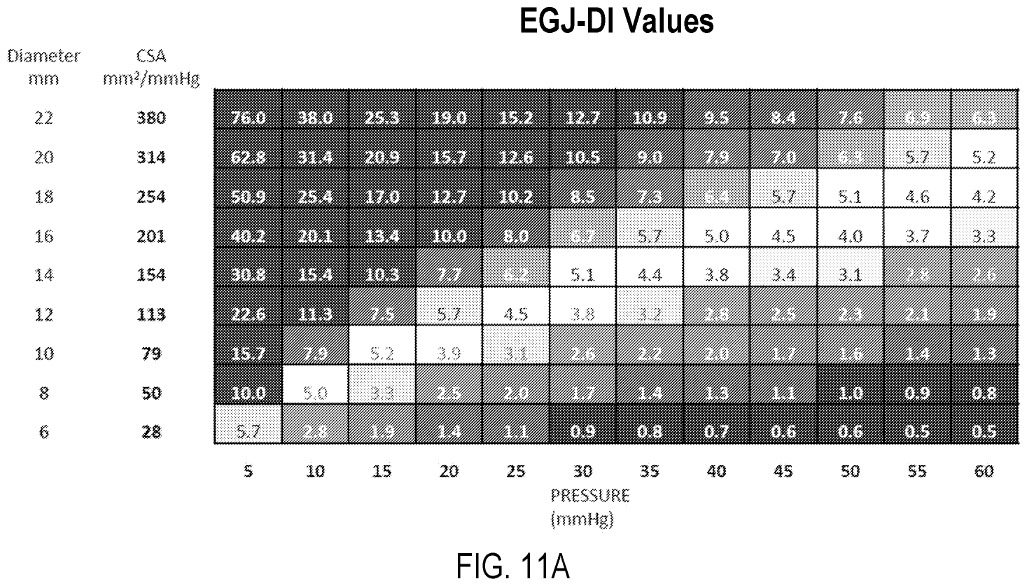

In FIG. 10, the entries labeled as “+” indicate pathognomonic patterns (high PPV), the entries labeled as “+/−” indicate patterns that can be seen, the entries labeled as “−/+” indicate patterns that are rare, and the entries labeled as “−” are almost never seen (high NPV). Examples of pathognomonic patterns include the following: normal EGJ opening and RACs indicate normal motility, normal EGJ opening and ACR is associated with absent contractility and IEM, abnormal EGJ opening and ACR is associated with Type I or Type II achalasia, and abnormal EGJ opening and SCR is associated with Type III achalasia. Transition patterns include those with BDCR, which is associated with an early transition state of muscular function and loss of neurologic control; those with IDCR, which is associated with a late transition state of muscular function and loss of neurologic control; myogenic patterns; and neurogenic patterns. For example, myogenic patterns may include BDCR/IDCR (weak focal short with normal rate) to ACR (scleroderma or severe GERD), Type II to Type I (dilatation), or Type III to Type II (dilatation and chronic obstruction). Examples of neurogenic patterns may include BDCR to SCR/Type III; BDCR/IDCR (chaotic with rapid rate) to Type III with RRCs; and Type III to Type II due to loss of excitatory neurons. Rule outs (i.e., high NPV) can include RACs that do not have achalasia and/or ACR without normal peristalsis or Type III achalasia.

Referring again to FIG. 5, one or more neural networks (or other suitable machine learning algorithms) are trained on the training data, as indicated at step 504. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). Training data can then be input to the initialized neural network, generating output as classified feature data. The quality of the classified feature data can then be evaluated, such as by passing the classified feature data to the loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. When the error has been minimized (e.g., by determining whether an error threshold or other stopping criterion has been satisfied), the current neural network and its associated network parameters represent the trained neural network. Different types of training algorithms can be used to adjust the bias values and the weights of the node connections based on the training examples. The training algorithms may include, for example, gradient descent, Newton's method, conjugate gradient, quasi-Newton, Levenberg-Marquardt, among others.

The one or more trained neural networks are then stored for later use, as indicated at step 506. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

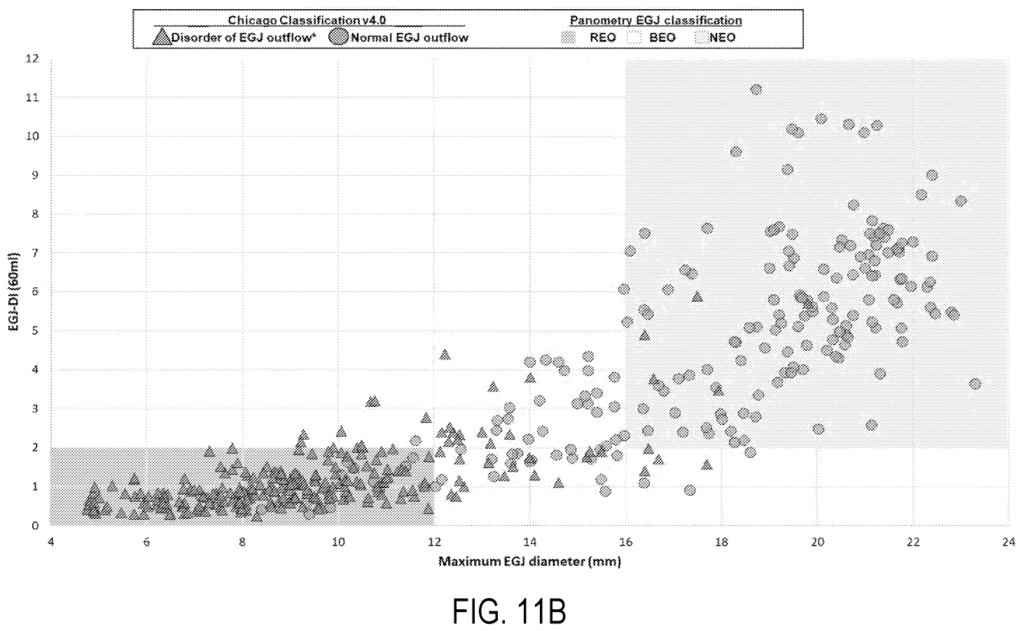

In addition to training neural networks, other machine learning or classification algorithms can also be trained and implemented for generating classified feature data. As one example, esophageal measurement data can be classified by computing parameters from the esophageal measurement data and classifying the esophageal measurement data based in part on those computed parameters. For instance, esophagogastric junction (“EGJ”) distensibility index (“EGJ-DI”) can be computed and used to classify esophageal measurement data. The EGJ-DI can be computed as,

$$E G J - D I = \frac{Narrowest {CSA}_{E G J}}{Intra - balloon Pressure} \qquad ( 1 )$$

where Narrowest CSA_{EGJ} is the narrowest cross-sectional area of the EGJ measured in the esophageal measurement data. An example table of EGJ-DI values is shown in FIG. 11A and an example association of FLIP Panometry EGJ opening parameters with EGJ obstruction based on a Chicago Classification v4.0 is shown in FIG. 11B. The association shown in FIG. 11B can advantageously be used to assess EGJ opening dynamics in the context of peristalsis based in part on balancing EGJ-DI and maximum EGJ diameter measurements.

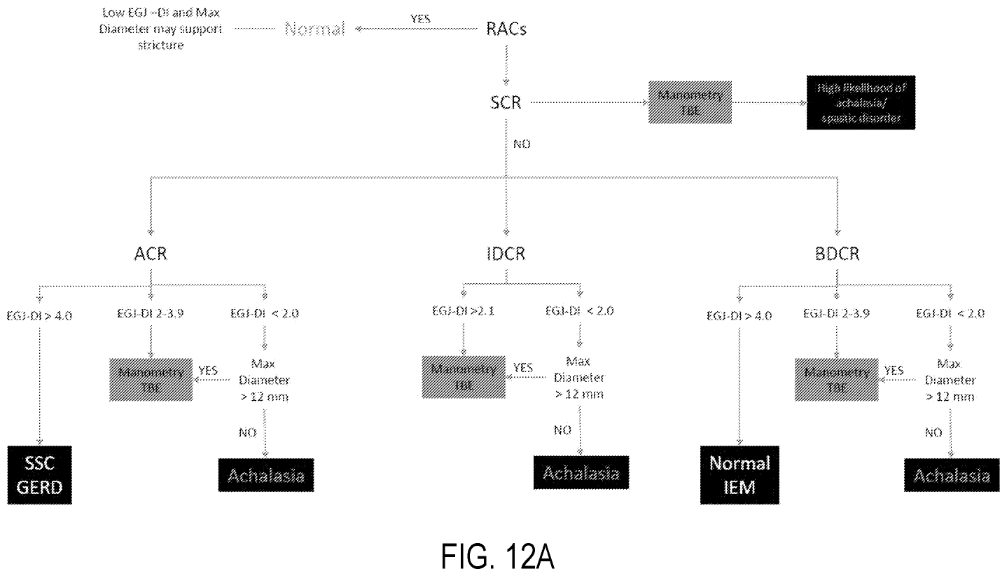

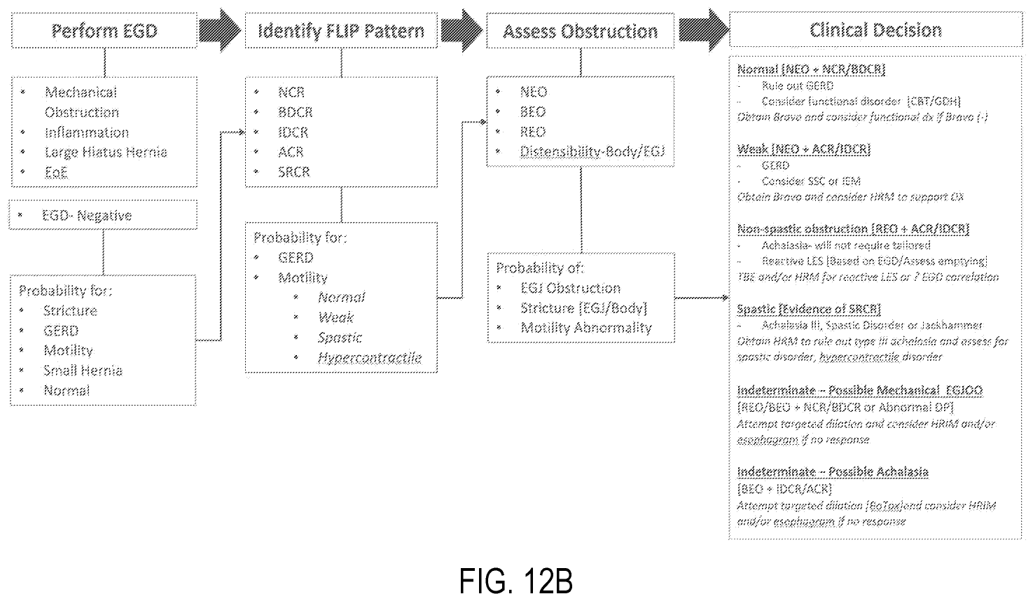

An example classification scheme based on EGJ-DI is shown in FIG. 12A. In the illustrated embodiment, the esophageal measurement data are first processed by the AI-based classifier to identify or otherwise determine the presence of any RACs in the esophageal measurement data. If no RACs are identified, then the esophageal measurement data can be classified as normal. If an SCR pattern is identified, then further imaging or testing of the subject can be recommended as an indication in the classified feature data, which may also indicate that the esophageal measurement data are representative of a high likelihood of achalasia and/or spastic disorder. If SCR patterns are not present, then an EGJ-DI value can be computed, estimated, or otherwise determined from esophageal measurement data and used as an input for an AI-based classifier. Depending on the identified RAC pattern(s) in the esophageal measurement data, different classifications of the esophageal measurement data can be implemented based on the EGJ-DI value and/or other data (e.g., maximum diameter indicated in the esophageal measurement data).

An example workflow for implementing a classification scheme according to some embodiments described in the present disclosure is shown in FIG. 12B. First, EGD is performed. If the EGD is negative, then FLIP can be used to obtain esophageal measurement data, which can then be processed with an AI-based classifier to identify RAC patterns and/or classify the esophageal measurement data as described above. The nature of any obstruction can be assessed based on the classified feature data (and/or findings from the EGD) and reviewed by a clinician to help inform their clinical decision making process.

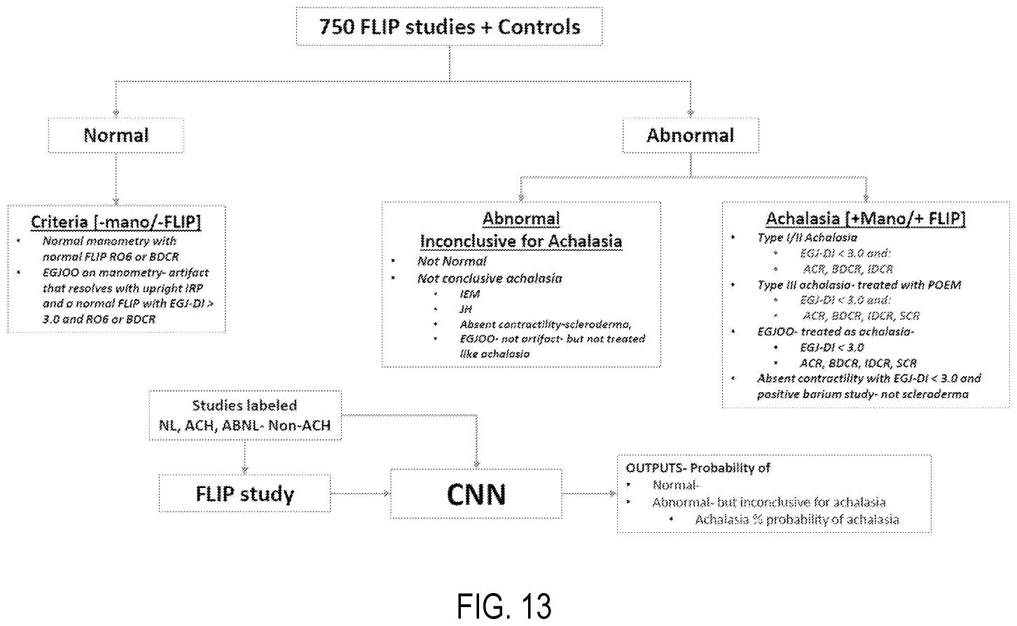

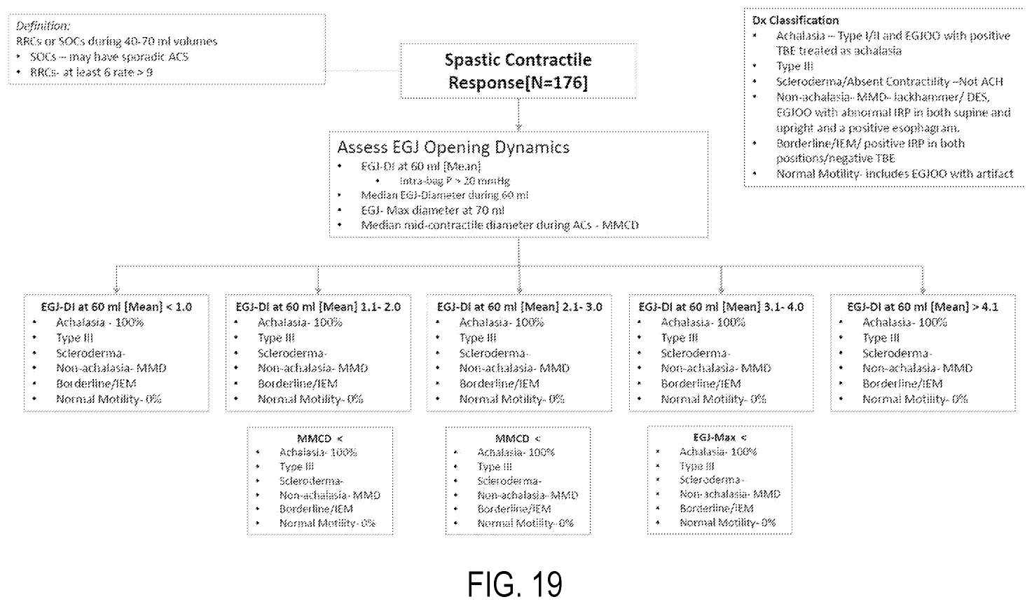

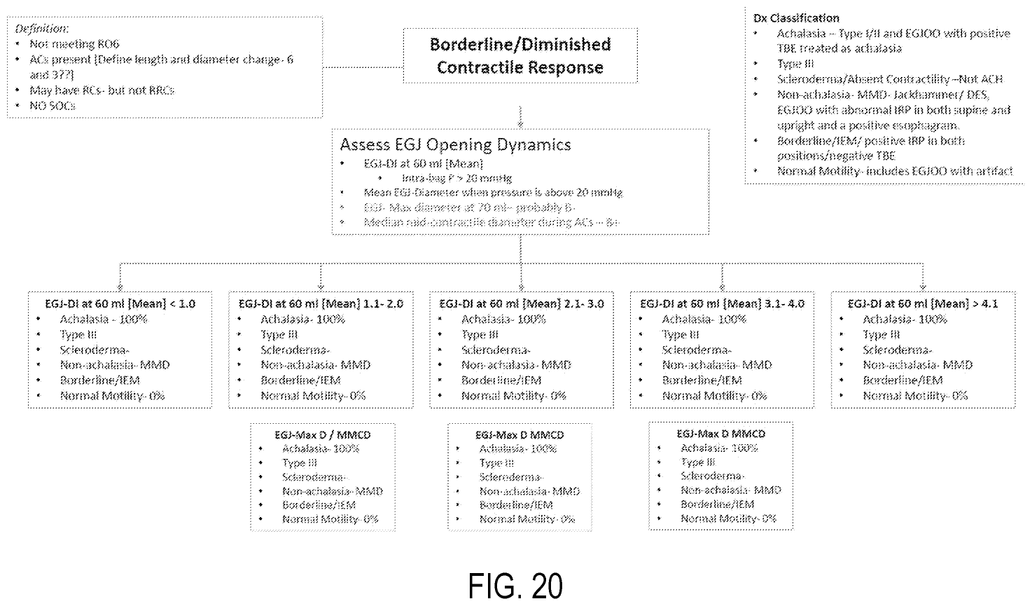

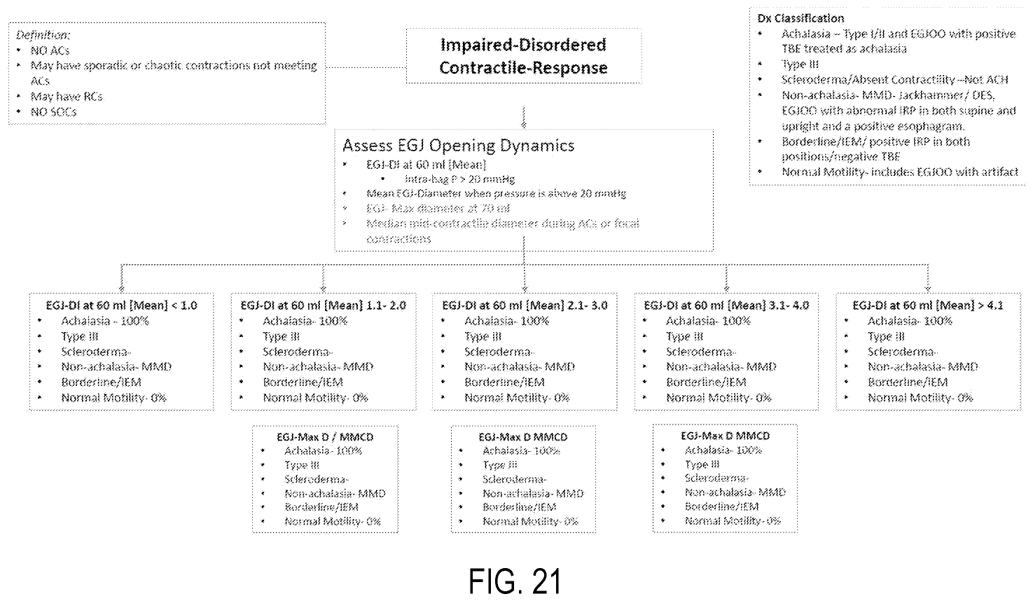

Additional example classification schemes that utilize both EGJ-DI (and/or other measured parameters) and contractile response patterns are shown in FIGS. 13-25. For example, in FIG. 13, a CNN is used as the AI-based classifier, which takes FLIP data as an input and outputs classified feature data indicating a probability that the FLIP data are indicative of a normal condition, an abnormal but inconclusive for achalasia condition, or an abnormal and percent probability of achalasia condition.

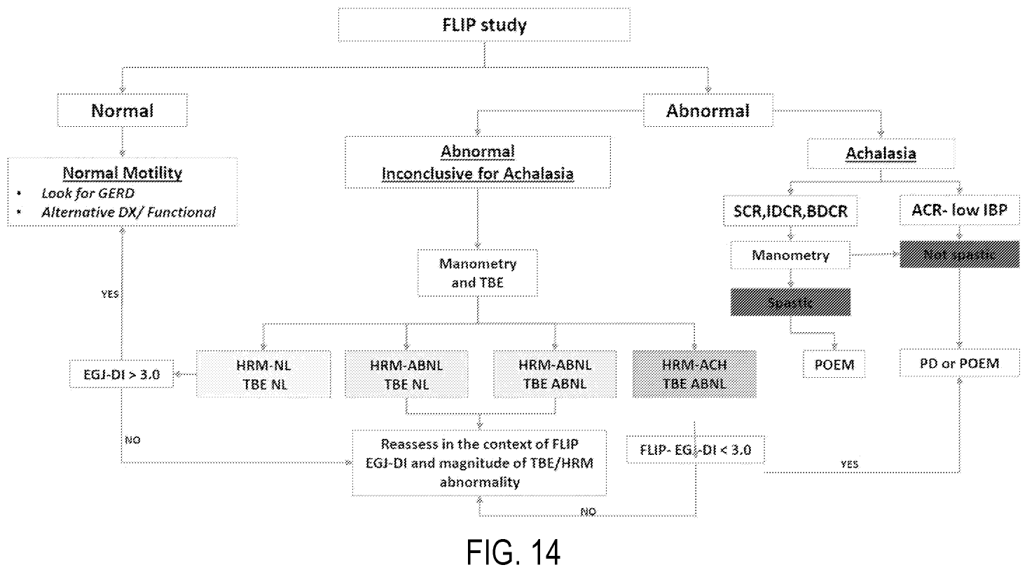

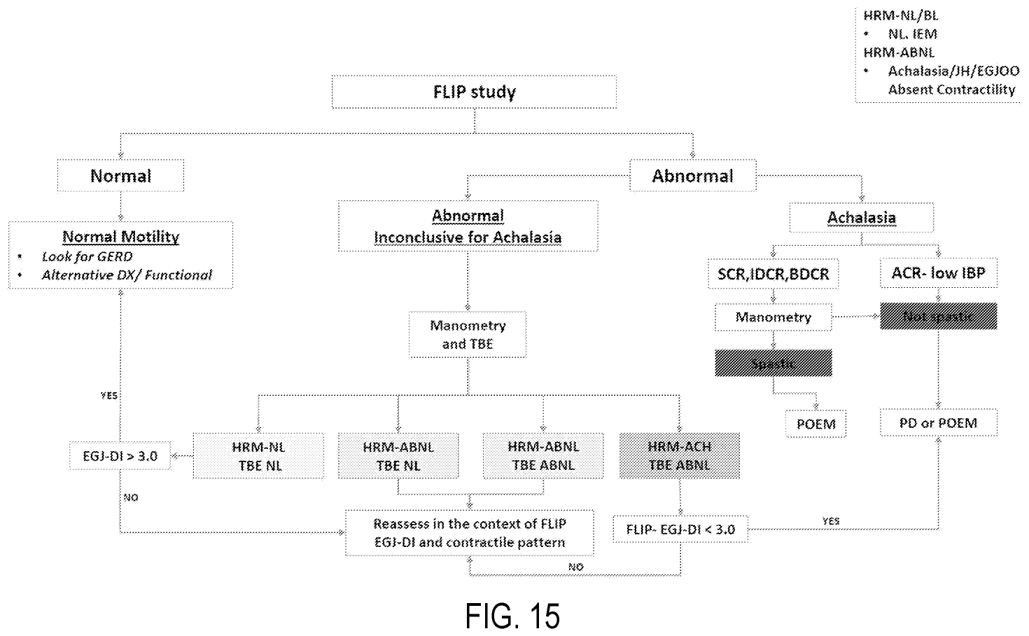

FIGS. 14 and 15 illustrate example an classification scheme in which FLIP data are processed by an AI-based classifier to generate classified feature data indicating a normal condition, an abnormal but inconclusive for achalasia condition, or an achalasia condition. When classified as the abnormal but inconclusive for achalasia condition, the classified feature data can include a recommendation for follow up manometry and/or TBE of the subject, or for classification of previously collected manometry and/or TBE data. These data can then be processed together with EGJ-DI values to either reclassify the data as indicating a normal condition or as recommending reassessment in the context of FLIP EGJ-DI and magnitude of TBE/HRM abnormality, as indicated in FIG. 14, or in the context of FLIP EGJ-DI and contractile patterns, as indicated in FIG. 15. Similarly, when classified as an achalasia condition, the classified feature data can further indicate one or more subconditions or class labels (e.g., spastic, not-spastic, PEOM, and/or PD) based on the RAC patterns identified in the FLIP data and/or based on manometry data.

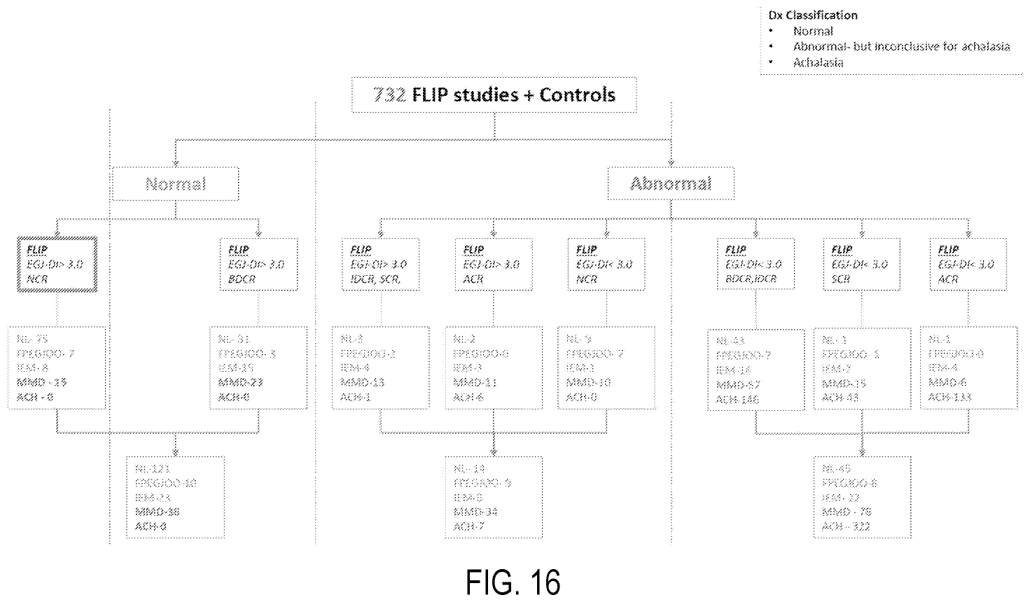

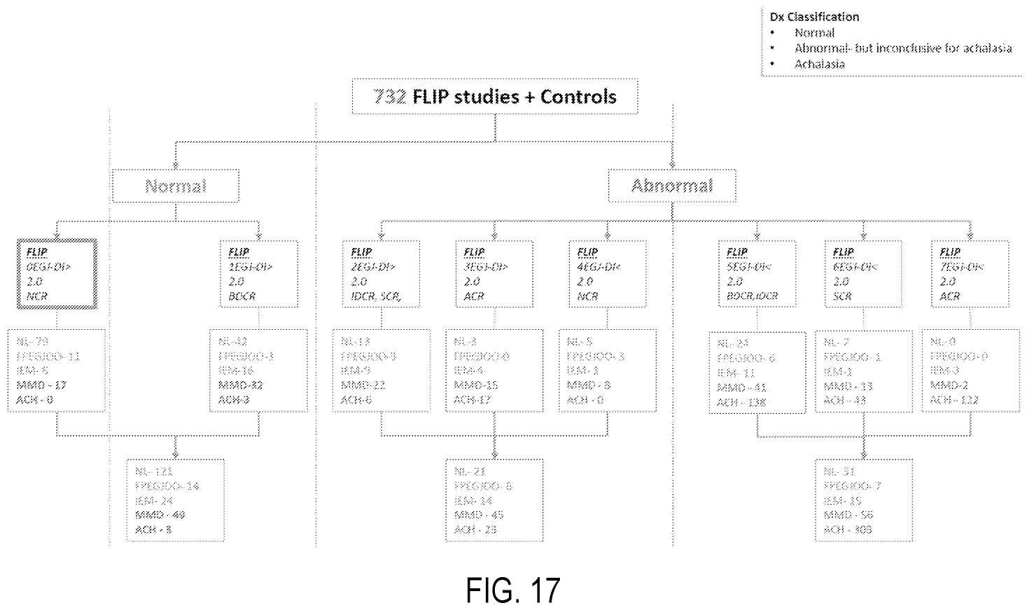

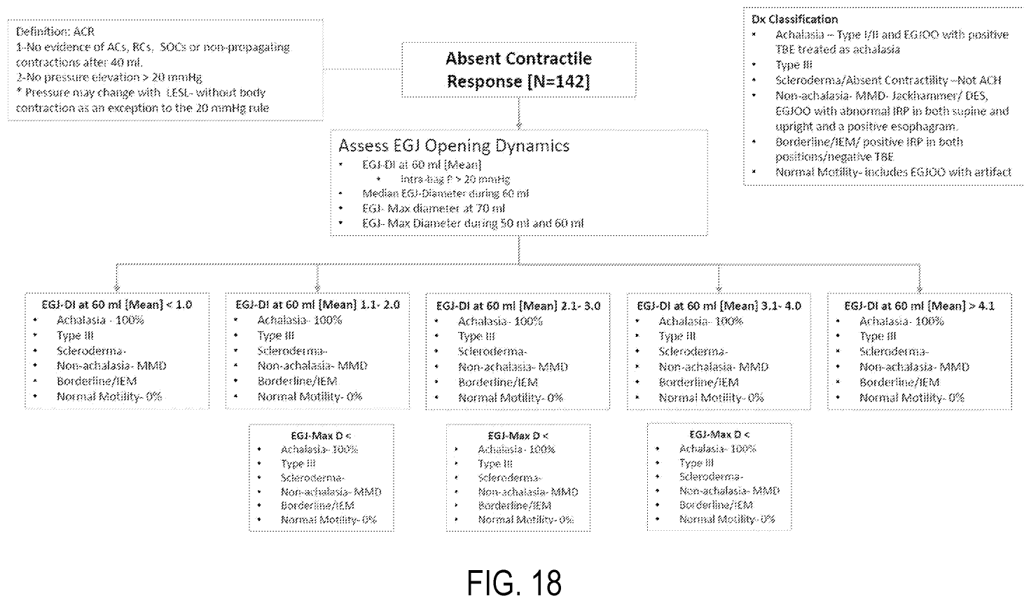

FIGS. 16 and 17 illustrate example classification schemes based on EGJ-DI and contractile patterns identified in the esophageal measurement data. FIGS. 18-21 illustrate example classification schemes based on contractile patterns identified in the esophageal measurement data and other parameters, such as EGJ-DI at 60 mL (mean), intra-bag pressure, median EGJ-DI during 60 mL, EGJ maximum diameter at 70 mL, EGJ maximum diameter during 50 mL and 60 mL, MMCD during ACs, and the like.

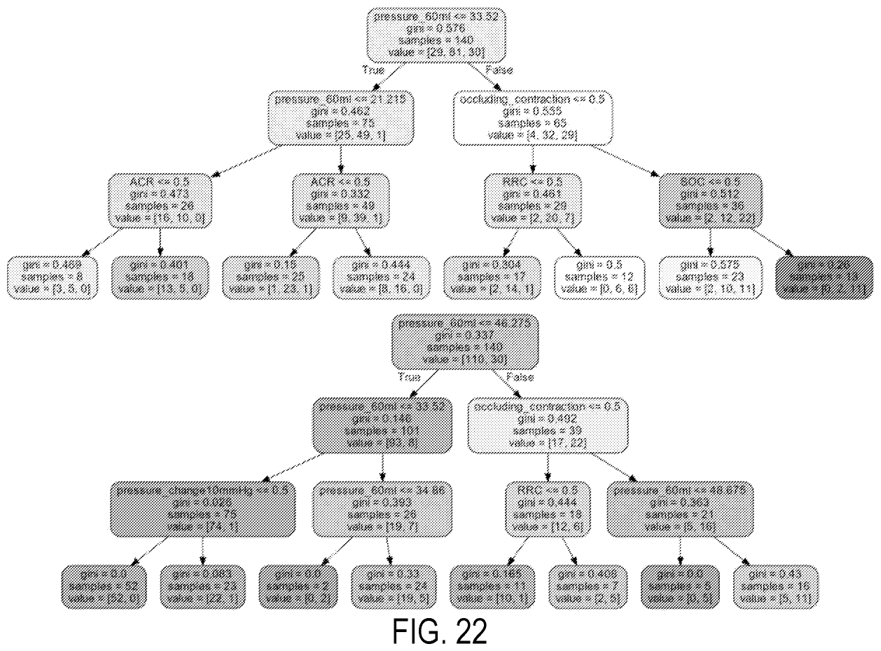

FIG. 22 illustrates an example random forest classification scheme.

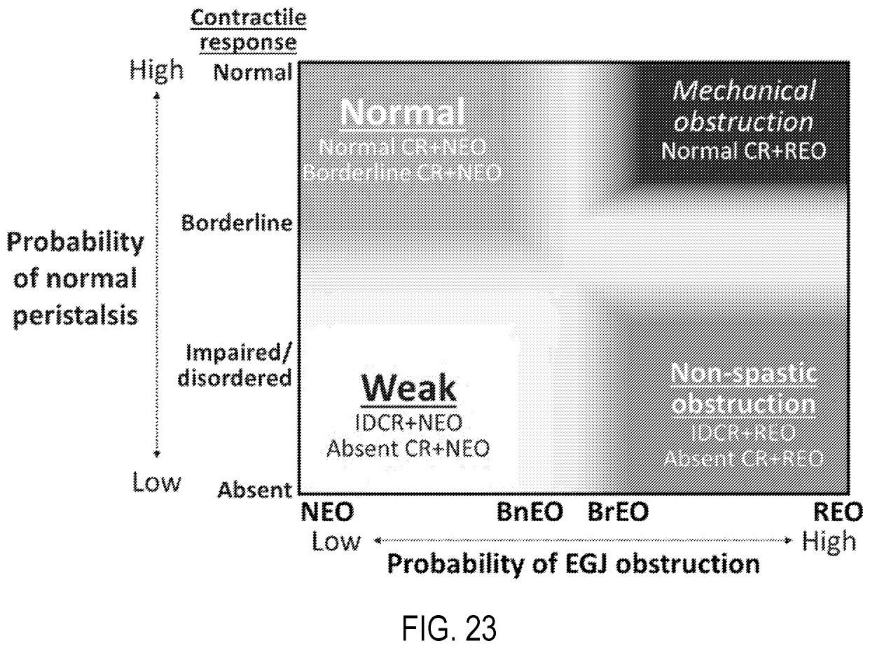

FIG. 23 illustrates an example classification scheme of esophageal motility. In the illustrated embodiment, a combination of FLIP panometry contractile response pattern and EGJ opening classification is applied to classify esophageal motility. Findings associated with clinical uncertainty (i.e., gray zones) can be classified as inconclusive. As a non-limiting example, an AI-based classifier implementing a support vector machine can be utilized to classify the contractile patterns identified in the esophageal measurement data and the EGJ opening data. In such embodiments, the computer system (e.g., computing device 150, server 152) may receive inputs such as esophageal measurement data, or as already identified contractile pattern data and EGJ opening data. The computer system executing the AI classification program, algorithm, or model then defines a margin using combinations of some of the input variables (e.g., contractile pattern, EGJ opening) as support vectors to maximize the margin. The margin corresponds to the distance between the two closest vectors that are classified differently. For example, the margin corresponds to the distance between a vector representing a first type of esophageal motility and a vector that represents a second type of esophageal motility.

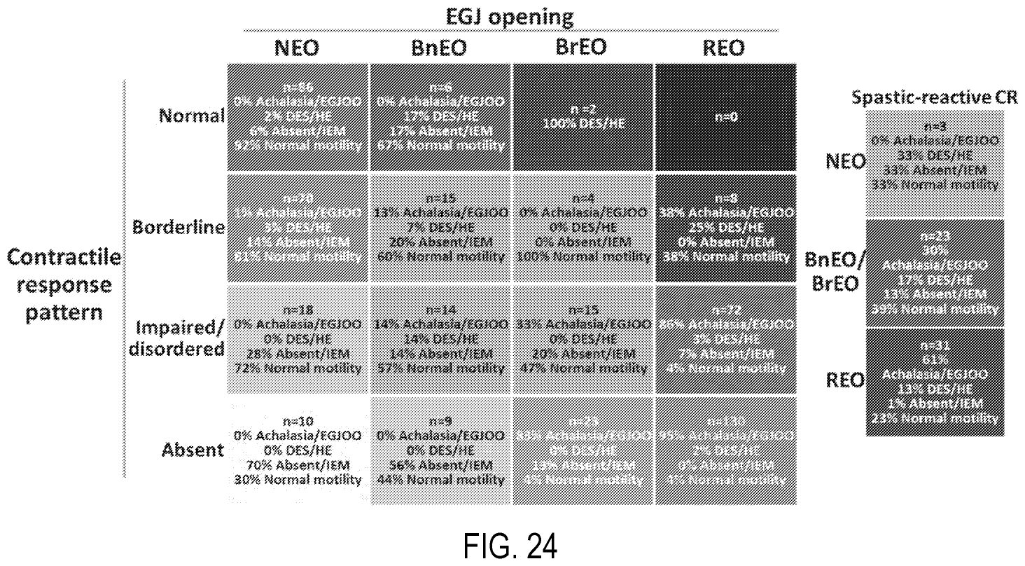

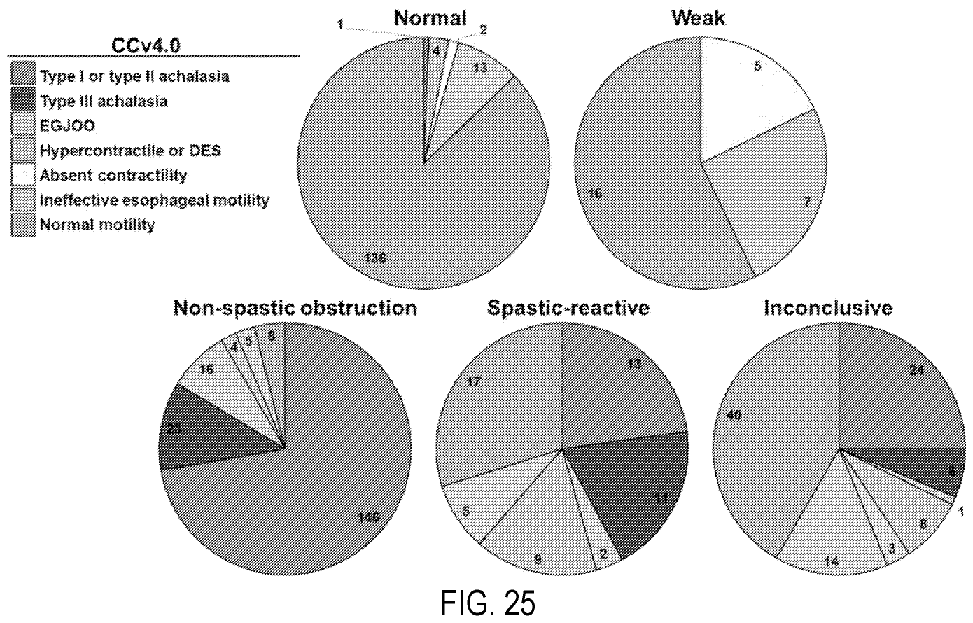

FIG. 24 illustrates an association between FLIP Panometry findings and Chicago Classification v4.0 (CCv4.0) high-resolution manometry (“HRM”) diagnoses. The number of patients (n) and associated diagnoses per CCv4.0 are shown in each box. FIG. 25 illustrates CCv4.0 diagnoses among FLIP panometry motility classifications. Each pie chart represents a FLIP panometry motility classification with proportions of conclusive CCv4.0 diagnoses (which are grouped by similar features for display purposes). Data labels represent number of patients.

The present disclosure has described one or more preferred embodiments, and it should be appreciated that many equivalents, alternatives, variations, and modifications, aside from those expressly stated, are possible and within the scope of the invention.

1. A method for generating classified feature data indicative of an upper gastrointestinal disorder in a subject based on esophageal measurement data acquired from the subject's esophagus, the method comprising:
(a) accessing esophageal measurement data with a computer system, wherein the esophageal measurement data comprise measurements of pressure within the subject's esophagus and changes in a geometry of the subject's esophagus;
(b) accessing a trained machine learning algorithm with the computer system, wherein the trained machine learning algorithm has been trained on training data in order to generate classified feature data from esophageal measurement data;
(c) applying the esophageal measurement data to the trained machine learning algorithm using the computer system, generating output as classified feature data that classify the esophageal measurement data as being indicative of an upper gastrointestinal disorder in the subject.

2. The method of claim 1, wherein the trained machine learning algorithm comprises a neural network.

3. The method of claim 2, wherein the neural network is a convolutional neural network.

4. The method of claim 1, wherein the training data include labeled data comprising esophageal measurement data labeled as corresponding to a contractile response pattern.

5. The method of claim 4, wherein the contractile response pattern comprises a distention-induced contractile response pattern.

6. The method of claim 5, wherein the distention-induced contractile response pattern comprises at least one of a repetitive antegrade contractions (RAC) pattern, an absent contractile response (ACR) pattern, a repetitive retrograde contractions (RRC) pattern, an impaired or disordered contraction (IDCR) pattern, or a spastic contractile (SCR) pattern.

7. The method of claim 6, wherein the SCR pattern comprises at least one of a sustained occluding contraction (SOC) pattern or a sustained LES contraction (sLESC) pattern.

8. The method of claim 1, wherein the trained machine learning algorithm is trained on the training data in order to identify a contractile response pattern in the esophageal measurement data and to generate the classified feature data based on the contractile response pattern identified in the esophageal measurement data.

9. The method of claim 8, wherein the contractile response pattern comprises a distention-induced contractile response pattern.

10. The method of claim 9, wherein the distention-induced contractile response pattern comprises at least one of a repetitive antegrade contractions (RAC) pattern, an absent contractile response (ACR) pattern, a repetitive retrograde contractions (RRC) pattern, an impaired or disordered contraction (IDCR) pattern, or a spastic contractile (SCR) pattern.

11. The method of claim 10, wherein the SCR pattern comprises at least one of a sustained occluding contraction (SOC) pattern or a sustained LES contraction (sLESC) pattern.

12. The method of claim 1, further comprising computing an esophagogastric junction distensibility index (EGJ-DI) value from the esophageal measurement data, and wherein step (c) also includes applying the EGJ-DI value to the trained machine learning algorithm in order to generate the output as the classified feature data.

13. The method of claim 1, wherein the trained machine learning algorithm comprises a random forest model.

14. The method of claim 1, wherein the esophageal measurement data comprise measurements of pressure within the subject's esophagus and changes in a diameter of the subject's esophagus and esophagogastric junction (EGJ).

15. The method of claim 14, wherein the esophageal measurement data are acquired from the subject using a functional lumen imaging probe.

16. The method of claim 1, wherein the classified feature data comprise a probability score representative of a probability that the esophageal measurement data are indicative of the upper gastrointestinal disorder in the subject.

17. A method for generating a report that classifies an upper gastrointestinal disorder in a subject, the method comprising:
(a) accessing functional lumen imaging probe (FLIP) data with a computer system, wherein the FLIP data depict esophageal pressure and diameter measurements in the subject's esophagus;
(b) accessing a trained classification algorithm with the computer system;
(c) generating classified feature data with the computer system by inputting the FLIP data to the trained classification algorithm, generating output as the classified feature data, wherein the classified feature data classify the FLIP data as being indicative of an upper gastrointestinal disorder in the subject; and
(d) generating a report from the classified feature data using the computer system, wherein the report indicates a classification of the FLIP data being indicative of the upper gastrointestinal disorder in the subject.

18. The method of claim 17, further comprising computing an esophagogastric junction distensibility index (EGJ-DI) value from the FLIP data, and wherein step (c) also includes applying the EGJ-DI value to the trained classification algorithm in order to generate the output as the classified feature data.

19. The method of claim 17, wherein the trained classification algorithm comprises a random forest model.

20. The method of claim 17, wherein the FLIP data comprise measurements of pressure within the subject's esophagus and changes in a diameter of the subject's esophagus and esophagogastric junction (EGJ).

21. The method of claim 17, wherein the classified feature data comprise a probability score representative of a probability that the FLIP data are indicative of the upper gastrointestinal disorder in the subject.
